(19) United States
(12) Patent Application Publication
(10) Pub. No.: US 2021/0000904 A1
(43) Pub. Date: Jan. 7, 2021

(54) NOVEL USE OF MAYDIS STIGMA EXTRACT IN PROPHYLAXIS AND THERAPY OF SLEEP DISORDER

(71) Applicants: KONKUK UNIVERSITY GLOBAL INDUSTRY-ACADEMIC COLLABORATION FOUNDATION, Chungju-si (KR); NEUROVENTI, Seoul (KR)

(72) Inventors: Chan Young SHIN, Seoul (KR); Seol Heui HAN, Seoul (KR); Kyoung Ja KWON, Seoul (KR); Ryoung Eun KIM, Ulsan (KR); So Hyun JOO, Seoul (US)

(21) Appl. No.: 15/733,584

(22) PCT Filed: Mar. 7, 2019
(86) PCT No.: PCT/KR2019/002640

§ 371 Date: Sep. 4, 2020

(30) Foreign Application Priority Data
Mar. 7, 2018 (KR) 10-2018-0026995

(51) Int. Cl. A61K 36/899; A61P 25/20

(52) U.S. Cl. CPC A61K 36/899; A61K 2236/333; A61K 2236/331; A61P 25/20

(57) ABSTRACT

The present invention provides a novel use of a Maydis stigma extract in prophylaxis, alleviation, or therapy of sleep disorder. A Maydis stigma extract has the effect of reducing sleep onset latency and increasing sleep duration, as high as or higher than the effect of ketamine or pentobarbital, which are conventionally used as soporifics. As a natural substance, the Maydis stigma extract does not cause side effects, such as cytotoxicity, resistance, or dependency, even when used for a long term.

### CROSS-REFERENCE TO RELATED APPLICATIONS

This application is a national phase under 35 U.S.C. § 371 of International Application No. PCT/KR2019/002640, filed Mar. 7, 2019, which claims priority to and benefit of Korean Patent Application No. 2018-0026995, filed on Mar. 7, 2018, the entire contents of each of which are incorporated herein by reference in their entirety.

### FIELD OF THE DISCLOSURE

The present invention relates to a novel use of a Maydis stigma extract in prophylaxis or therapy of sleep disorder.

### DESCRIPTION OF RELATED ART

Sleep is a naturally occurring state in which changes of consciousness, suppression of sense activation, and suppression of all of voluntary muscles happen. As an activity that restores the energy of body and provides rest, it is a physiological phenomenon that is very important for maintaining health and well-being. During sleep, most of the body system turns into an anabolic state, which is a state where the recovery and growth of the immunological system, nervous system and musculoskeletal system are reduced. In modern society, however, many people are experiencing sleep disorder caused by aging, stress, anxiety and restlessness, or caused by or as a result of neurological diseases such as Alzheimer's disease, Parkinson's disease, autism spectrum disorders (ASD), attention deficit hyperactivity disorder (ADHD) and autism. Sleep disorder is a concept including a state in which it is difficult to maintain alertness during the day when healthy sleep is not achieved or even when enough sleep is achieved, or a state in which difficulty is experienced when asleep or awake because sleep rhythm is disrupted, and there are various symptoms in patients such as insomnia, narcolepsy, restless legs syndrome, sleep apnea syndrome and the like. Sleep disorder may become causes of learning disability and a decrease in work efficiency, various safety accidents such as traffic accidents and the like, emotional disorder, social adjustment disorder and the like, and when it is not adequately treated, medical, neurological and psychiatric diseases may worsen, or recovery may be delayed.

Disease condition due to sleep deprivation is similar to various phenomena shown in aging, neurological diseases such as Alzheimer's disease, Parkinson's disease or the like, alcohol addiction and the like. On the contrary, there are reports that sleep disorder occurs as a common symptom of neurological diseases, neuropsychiatric disorders such as Alzheimer's disease and the like, developmental disorders such as autism and the like, and aging. The progression of aging is very closely associated with changes in language ability, quantitative and qualitative changes of sleep, and changes in cognitive function. Particularly, in terms of sleep, various changes are shown in the amount of sleep and the quality of sleep such as slow wave sleep, spindle density, sleep continuity/fragmentation and the like.

The treatment of sleep disorder is largely classified into pharmacotherapy and non-pharmacological therapy, and even though pharmacotherapy by hypnotics and the like may be the easiest and fastest method, a method that minimizes drug use and combines cognitive-behavioral therapy is used recently due to adverse reaction risks depending on drug use, particularly, due to worries for the effect of long-term intake and drug resistance, and rebound and withdrawal symptoms when stopped. For hypnotics as medicine for severity and severe sleep disorders treatment, Z-drugs such as Zaleplon, Zopiclone and the like are recently used as GABA-A modulators in large quantity, and as a means to promote sleep by circadian rhythm, melatonin is used, which is capable of suppressing the activity of suprachiasmatic nucleus (SCN).

In recent years, with growing interest in inducing or maintaining sleep, many effective and safe novel hypnotics have been released. Drugs whose main ingredient is sleep hormone melatonin that has a different reaction mechanism from those of the conventional sleep disorder therapeutic agents are becoming commercially available domestically in Korea, and while these are expected as novel hypnotics that normalize the period of melatonin to induce sleep without hallucination or dependency, the current situation is that there are still great demands for high-efficiency, low-dependency and low side-effect sleep aids as anti-psychotic drugs under domestic regulations.

As a method of treating sleep disorder, bananas, potatoes, honey, onions, lettuces and the like are known as foods for sleep induction other than drug therapy, and while these are mainly thought to have relevance with the production of melatonin which is a sleep adjustment hormone, their specific mechanism is not known.

Even though Ecklonia cava (phlorotannin) is known as a sleep-inducing natural product that has been reported to induce activation of GABAA benzodiazepine, sleep aids based on natural products currently have limited market growth due to their effects and uncertainties of mechanism.

Maydis stigma is the style and stigma of corn (Zea mays L.), and it is called Okmisu in a fine thread or hair shape, and it is commonly referred as corn silk. As reported in ancient literatures such as the Compendium of Materia Medica and the like, the function of Maydis stigma is known to have medicinal effects when used for urinary disease treatment, diuretics, blood coagulation, diseases related to adult diseases and the like. In China, many people have used corn silk as a therapeutic agent because it is known to be effective in diabetes, and in Korea, it is also known to be good for stomach, organs and the like, and particularly, it has been widely used as folk remedy medicine for diabetes, hepatitis, urethral stone, high blood pressure, spitting of blood, hemoptysis, prevention of nose bleeding and the like, because it has a diuretic effect and a blood pressure lowering effect.

While Maydis stigma has been traditionally used as an edible raw material for folk remedy as mentioned above, ingredient identification and studies related thereto are very insufficient. Recently, interest in flavonoid-based substances contained in corn silk has grown, and patents related to maysin which is one of flavonoid substances are Korean Registered Patent No. 10-1817512, Korean Registered Patent No. 10-1778752 and the like.

However, there is no study related to a sleep improvement use of a Maydis stigma extract.

### SUMMARY OF THE INVENTION

The present inventors have spent efforts to develop natural products capable of preventing, alleviating or treating sleep disorder related to a melatonin receptor. As a result, the present inventors completed the present invention by confirming an increase in the expression of melatonin receptors, a sleep-inducing effect in a mouse model, and a sleep time increasing effect by a Maydis stigma extract.

Therefore, an object of the present invention is to provide a composition for preventing, alleviating or treating sleep disorder.

Another object of the present invention is to provide a method of preventing or treating sleep disorder, including a step of administering a composition comprising a Maydis stigma extract as an active ingredient to a subject.

Another object of the present invention is to provide a use of a composition for preventing or treating sleep disorder comprising a Maydis stigma extract as an active ingredient.

Other objects and advantages of the present invention become more specific by the description, claims and drawings of the invention below.

In order to solve the above-mentioned problems, the present invention provides a composition for preventing or treating sleep disorder, comprising a Maydis stigma extract as an active ingredient.

In addition, the present invention provides a health functional food composition for preventing or alleviating sleep disorder, comprising a Maydis stigma extract as an active ingredient.

In addition, the present invention provides a use of a composition for preventing and treating of sleep disorder comprising a Maydis stigma extract as an active ingredient.

By decreasing sleep onset time and increasing sleep time with protective effect against neuronal apoptosis in brain cell, the composition including the Maydis stigma extract of the present invention as an active ingredient has an effect of preventing, alleviating or treating sleep disorder, insomnia and the like effectively, which appear in anxiety, aging due to stress, neurodegenerative diseases such as Alzheimer's disease and neurodevelopmental disorders such as autism and attention deficit hyperactivity disorder.

In addition, the composition including the Maydis stigma extract of the present invention as an active ingredient can be applied in various products including food compositions and quasi-drug compositions for preventing or alleviating sleep disorder or insomnia as natural medicine.

In addition, since the Maydis stigma extract of the present invention does not show toxicity as a result of animal experiments and is derived from natural products, it can be continuously used safely without causing serious irritation in the body or causing toxic activity other than efficacy for preventing or alleviating sleep disorder or insomnia.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1A-B illustrates changes in the expression of melatonin receptors in neuronal cells treated with a Maydis stigma extract: FIG. 1A illustrates changes in the expression of melatonin receptors in neuronal cells treated with a Maydis stigma extract via RT-PCR analysis, and FIG. 1B is the result of quantifying changes in the expression of melatonin receptors;

FIG. 2A-B illustrates sleep-inducing and sleep-promoting effects by a Maydis stigma extract in experimental animals: 2A is the result of confirming sleep onset time, and 2B is the result of confirming sleep duration time;

FIG. 3A-B illustrates toxicity of a Maydis stigma extract in experimental animals: FIG. 3A illustrates weight changes, and 3B show changes in organ weights;

FIG. 4 illustrates the protective effects of a Maydis stigma extract in cortical neuronal cells.

FIG. 5A-B illustrates sleep-inducing and sleep-promoting effects depending on concentrations of a Maydis stigma extract in experimental animals: 5A is the result of confirming sleep onset time, and 5B is the result of confirming sleep duration (SM is an administration group for a Maydis stigma extract, and VR is an administration group for Valeriana fauriel Briquet);

FIG. 6A-B illustrates toxicity depending on concentrations of a Maydis stigma extract in experimental animals: FIG. 6A illustrates weight changes, and FIG. 6B shows changes in organ weights;

FIG. 7A-B illustrates changes in the expression of melatonin receptors in experimental animals depending on concentrations of a Maydis stigma extract: FIG. 7A illustrates changes in the expression of melatonin receptors in cerebral cortex, and FIG. 7B illustrates changes in the expression of melatonin receptors in hypothalamus (SM is an administration group for a Maydis stigma extract, and VR is an administration group for Valeriana fauriel Briquet); and

FIG. 8A-B illustrates sleep-inducing and sleep-promoting effects in a sleep deprivation model depending on concentrations of a Maydis stigma extract: 8A is the result of confirming sleep onset time, and 8B is the result of confirming sleep duration (SM is an administration group for a Maydis stigma extract, and VR is an administration group for Valeriana fauriel Briquet).

### DETAILED DESCRIPTION OF THE INVENTION

Hereinafter, exemplary embodiments of the present disclosure will be described in detail to be embodied by those skilled in the art. The present disclosure may be implemented in various forms and is not limited to the following embodiments.

As described above, even though Maydis stigma has been traditionally used as an edible raw material of folk remedy for diabetes, hepatitis, urethral stone, high blood pressure, spitting of blood, hemoptysis, prevention of nose bleeding and the like, the sleep improving effect of a Maydis stigma extract has not been reported yet.

Since the Maydis stigma extract according to the present invention exhibits an effect of an increase in the expression of melatonin receptors, a sleep-inducing effect in mouse models, a sleep time increasing effect, a neuron protective effect in neuronal cell death models, a sleep-inducing effect in a sleep deprivation model, and a sleep time increasing effect, a Maydis stigma extract may be effectively used as an active ingredient of a composition for preventing, alleviating and treating sleep disorder.

The present invention provides a composition for preventing, alleviating or treating sleep disorder, including a Maydis stigma extract as an active ingredient.

As used here, the term ‘extract’ has a meaning of a crude extract, which is commonly used in the art as described above, and it also broadly includes a fraction which is additionally fractionated from an extract. That is, the Maydis stigma extract not only includes that which is obtained using an extraction solvent as described above, but also includes that which is obtained by additionally applying a purification process herein. For example, fractions that are obtained by passing the extract through an ultrafiltration membrane having a certain molecular weight cut-off value and fractions that are obtained by various purification methods that are additionally performed such as separation by various chromatographs (manufactured for separation by size, charge, hydrophobicity or affinity) and the like are also included in the natural product extract of the present invention.

When a Maydis stigma extract used in the composition of the present invention is obtained by treating an extraction solvent to the Maydis stigma, various extraction solvents may be used. According to one embodiment of the present invention, a polar solvent or a nonpolar solvent may be used. Examples of suitable polar solvents include (i) water, (ii) alcohol (preferably, methanol, ethanol, propanol, butanol, normal-propanol, iso-propanol, normal-butanol, 1-pentanol, 2-butoxyethanol or ethylene glycol), (iii) acetic acid, (iv) dimethyl-formamide (DMFO), and (v) dimethyl sulfoxide (DMSO). Examples of suitable nonpolar solvents include acetone, acetonitrile, ethyl acetate, methyl acetate, fluoroalkane, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethyl amine, ether, carbon tetrachloride and THF.

According to another embodiment of the present invention, examples of the extraction solvent used in the present invention include (a) water, (b) anhydrous or hydrous lower alcohol of carbon number 1 to 4 (methanol, ethanol, propanol, butanol and the like), (c) a mixed solvent of the lower alcohol and water, (d) acetone, (e) ethyl acetate, (f) chloroform, (g) butyl acetate, (h) 1,3-butyleneglycol, (i) hexane and (j) diethyl ether. According to a specific embodiment of the present invention, the Maydis stigma extract of the present invention is obtained by treating Maydis stigma with water.

The Maydis stigma extract used in the present invention may be prepared in a powder state by an additional process such as vacuum distillation and freeze-drying, spray drying or the like.

As used herein, the term ‘comprising as an active ingredient’ means including a sufficient amount to achieve efficacy or activity of the Maydis stigma extract below. The present invention is a composition extracted from Maydis stigma which is a natural plant substance, and the quantitative upper limit at which the Maydis stigma extract is included in the composition of the present invention may be chosen within an appropriate range by those skilled in the art to carry out.

The composition of the present invention may be used in prophylaxis or therapy of illness, disease or condition related to various sleep disorders.

As used herein, the term “preventing, alleviating or treating sleep disorder” means to be directed to reduce sleep onset time and increase sleep duration.

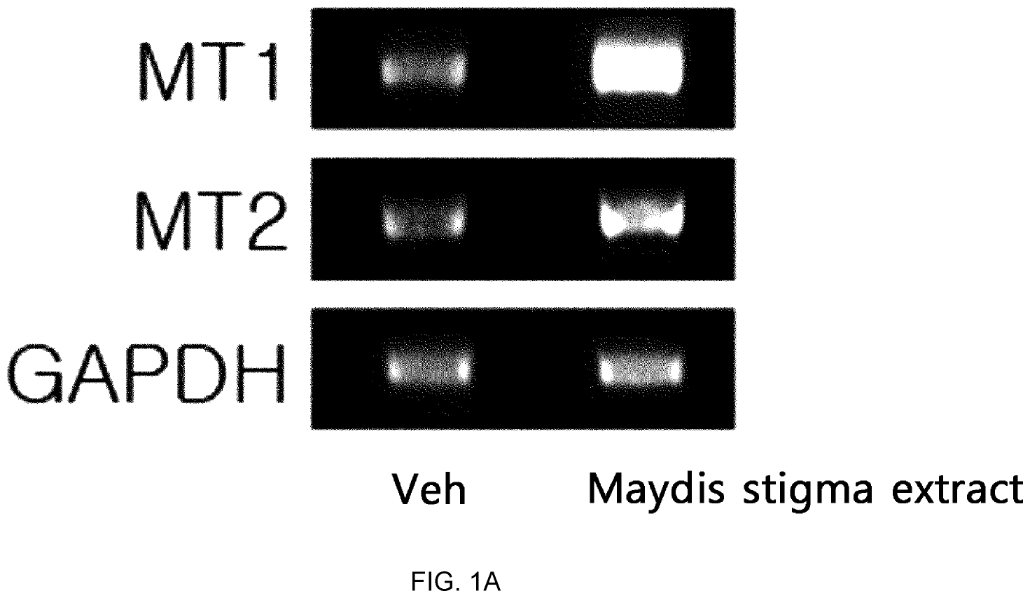

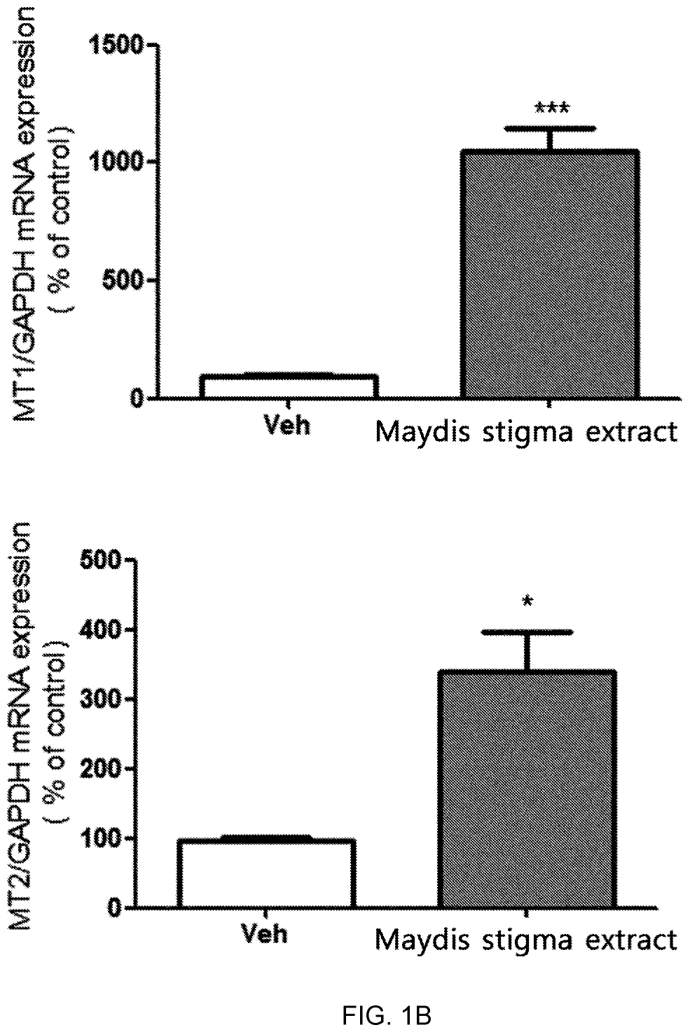

In a specific exemplary embodiment of the present invention, the present inventors determined changes of melatonin receptors by a Maydis stigma extract in neurons, and as a result, it was confirmed that the expression amounts of MT1 and MT2 which are melatonin receptor genes controlling sleep and circadian rhythm were increased. Specifically, the composition including the Maydis stigma extract of the present invention increases MT1 mRNA expression amounts by 100 times, and MT2 mRNA expression amounts by 3 times, compared to control groups (FIG. 1).

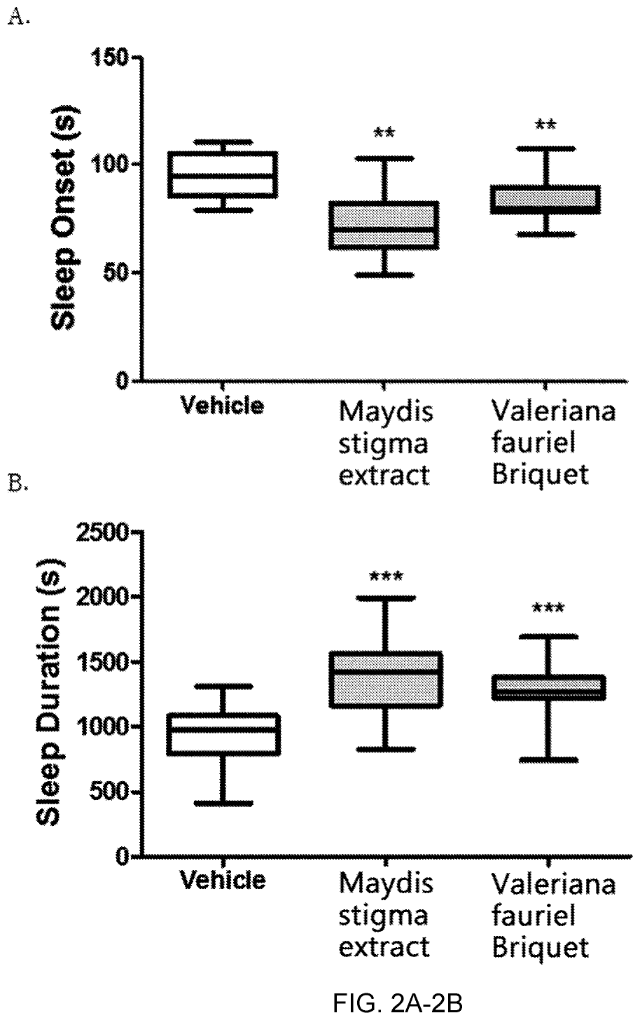

In addition, the present inventors determined the administration effect of a Maydis stigma extract in sleep-induced experimental animals, and as a result, a sleep-inducing effect was shown to be 30 seconds faster in a Maydis stigma extract administration group than in a negative control group, and it was confirmed that sleep onset time was decreased at an equivalent level or higher compared to a positive control group (FIG. 2).

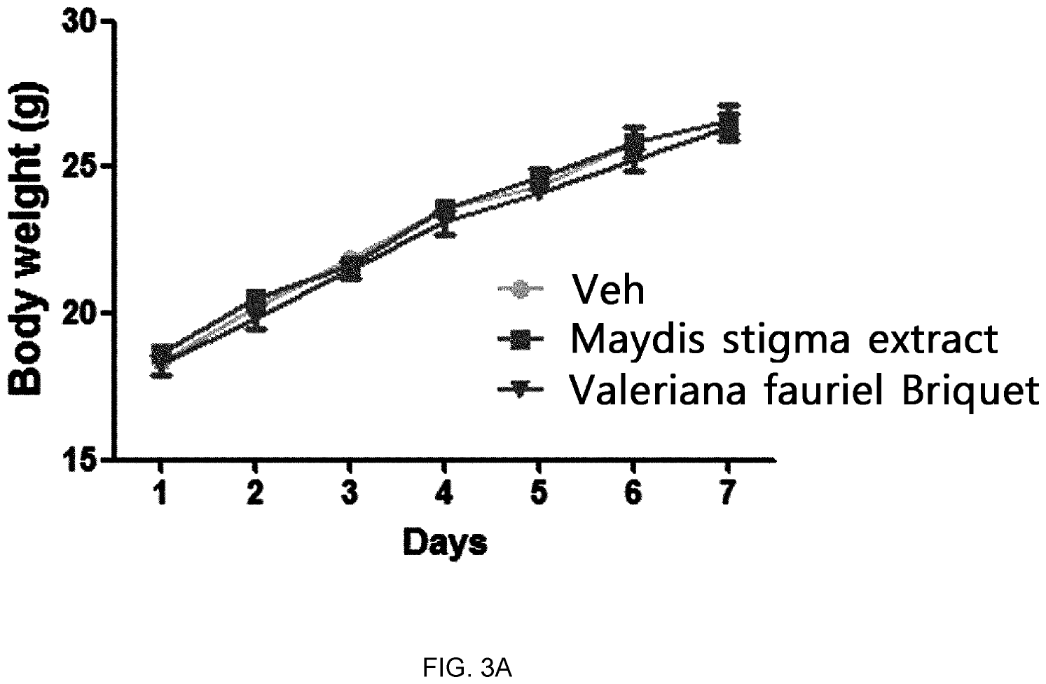

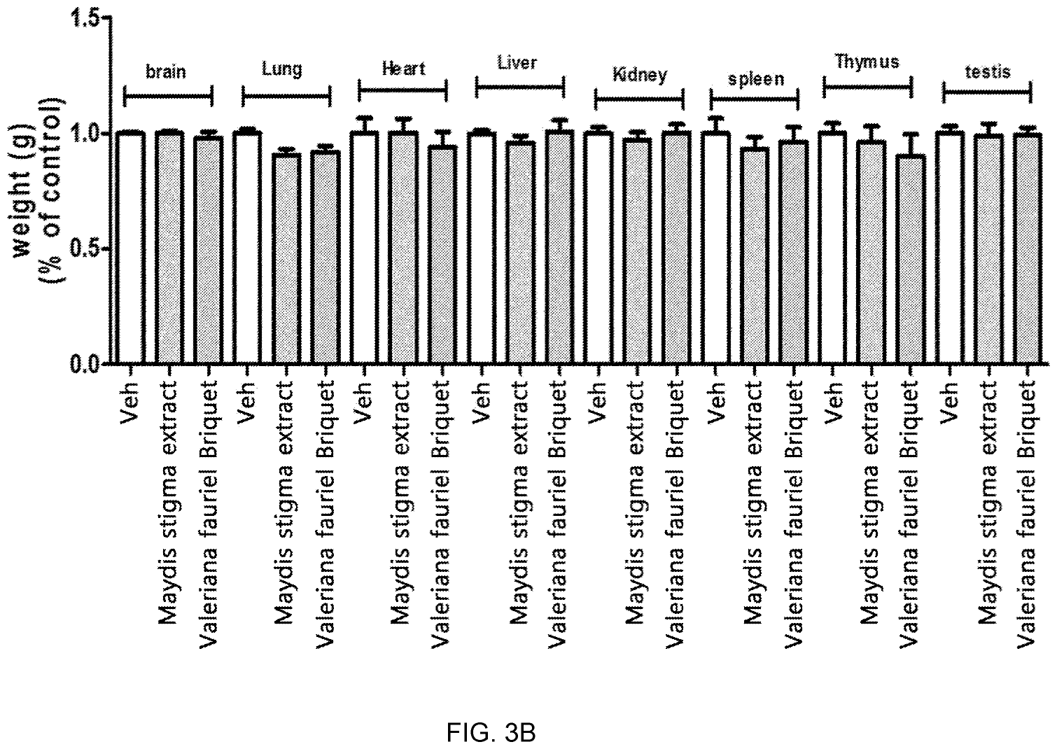

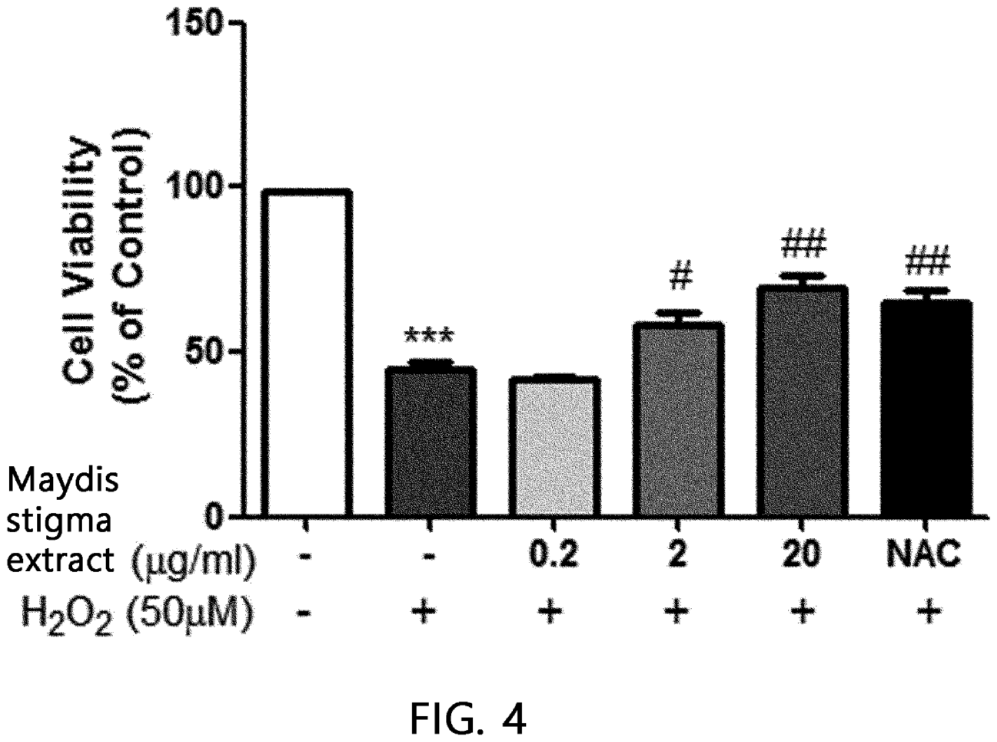

In addition, it was confirmed that in sleep-induced experimental animals, sleep disorder could be alleviated without cytotoxicity when an effective amount of the composition of the present invention was treated (FIG. 3), and when H2O2 was treated after pretreating the composition of the present invention to neurons, it was confirmed that neuronal cell death was suppressed (FIG. 4).

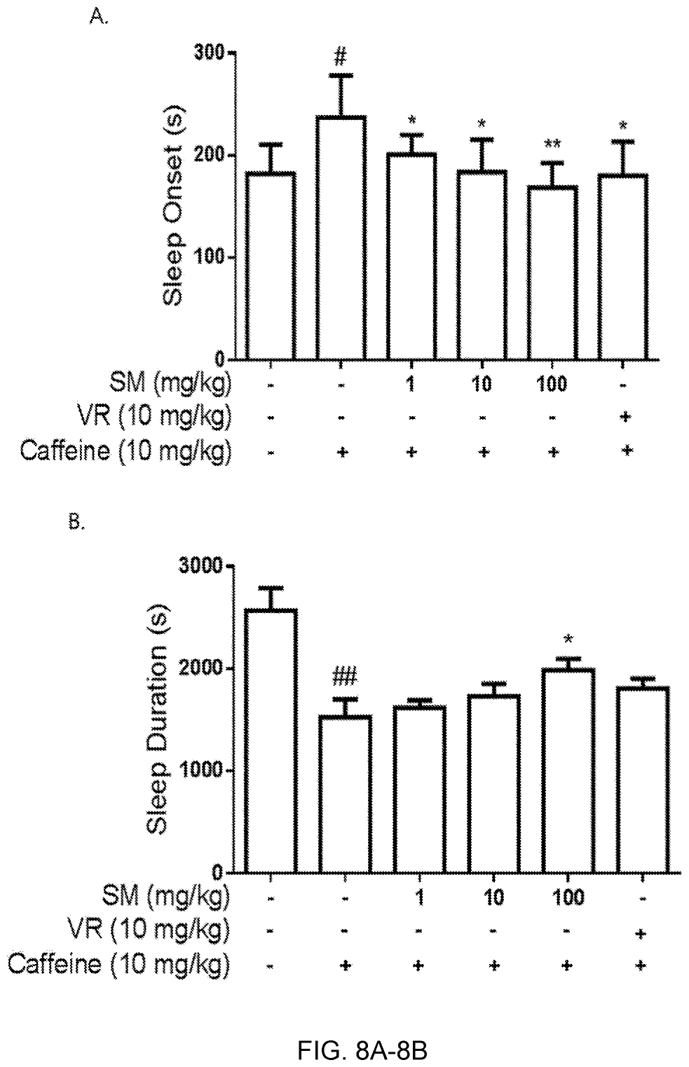

Further, in sleep-deprived experimental animal models by caffeine, sleep onset time was shortened in a concentration-dependent manner in the Maydis stigma extract administration group, and it was confirmed that sleep duration was increased (FIG. 8).

As such, the composition of the present invention may be prepared as a pharmaceutical composition.

According to a preferred embodiment of the present invention, the composition of the present invention is a pharmaceutical composition including (a) a pharmaceutically effective amount of the Maydis stigma extract of the present invention described above; and (b) a pharmaceutically acceptable carrier. As used herein, the term “pharmaceutically effective amount” refers to a sufficient amount to achieve efficacy or activity of the Maydis stigma extract described above.

When the composition of the present invention is prepared as a pharmaceutical composition, the pharmaceutical composition of the present invention includes a pharmaceutically acceptable carrier. The pharmaceutically acceptable carrier included in the pharmaceutical composition of the present invention is conventionally used upon formulation and examples include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, stearic acid, magnesium, mineral oil and the like, but are not limited thereto. The pharmaceutical composition of the present invention may additionally include a lubricating agent, a wetting agent, a sweetening agent, a flavoring agent, an emulsifying agent, a suspension, a preservative and the like, other than the above ingredients. The suitable pharmaceutically acceptable carrier and formulation are described in detail in Remington's Pharmaceutical Sciences (19th ed., 1995).

The pharmaceutical composition of the present invention may be administered orally or parenterally.

The suitable administration amount of the pharmaceutical composition of the present invention may be variously prescribed by considering factors such as a formulation method, manner of administration, age, weight, gender and diseased state of patients, food, administration time, administration route, excretion rate and response sensitivity. The general administration amount of the pharmaceutical composition of the present invention is within a range of 1 mg/kg to 100 mg/kg based on adults.

The pharmaceutical composition of the present invention may be prepared according to a method that can be easily carried out by those skilled in the art to which the corresponding invention pertains, by formulating using a pharmaceutically acceptable carrier and/or excipient to be prepared in a unit dosage form or placed in a multi-volume container. In this case, the dosage form may be in the form of a solution, a suspension, a syrup or an emulsion in an oil or aqueous medium, or in the form of an extract, a powder, a powdered drug, a granule, a tablet or a capsule, and may additionally include a dispersing agent or a stabilizing agent.

The composition of the present invention may be provided as a health functional food composition.

As used herein, the term “health functional food” refers to food that is prepared and processed in the form of a tablet, a capsule, a powder, a granule, a liquid, a pill and the like, using a raw material or ingredient having functionality useful in the human body. In this case, ‘functionality’ means controlling nutrients for the structure and function of the human body or obtaining a useful effect for sanitary use such as physiological action and the like. The health functional food of the present invention may be prepared according to a method conventionally used in the art, and when prepared, it may be prepared by adding a raw material and an ingredient that are conventionally added in the art. In addition, the dosage form of the health functional food may be prepared without limitation as long as it is a dosage form acknowledged to be health functional food. The health functional food composition of the present invention has an advantage that there is no side effect and the like that may occur during long-term intake, because food is a raw material unlike generic medicine, and since portability is excellent, it may be taken as a supplement for increasing an effect that alleviates symptoms of sleep disorder.

When the composition for preventing, alleviating or treating sleep disorder including the Maydis stigma extract of the present invention as an active ingredient is prepared as a health functional food composition, it may include additional ingredients that are conventionally added upon food preparation, in addition to the Maydis stigma extract as an active ingredient, and for example, it may include proteins, carbohydrates, lipid, nutrients, seasonings and flavoring agents. Examples of carbohydrates described above are monosaccharides, for example, glucose, fructose and the like; disaccharides, for example, maltose, sucrose, oligosaccharide and the like; and polysaccharides, for example, conventional sugar such as dextrin, cyclodextrin and the like, and sugar alcohol such as xylitol, sorbitol, erythritol and the like. For flavoring agents, natural flavoring agents [thaumatin and stevia extract (for example, rebaudioside A, glycyrrhizin and the like)] and synthetic flavoring agents (saccharin, aspartame and the like) may be used. For example, when the food composition of the present invention is prepared as a drink, it may additionally include citric acid, high fructose corn syrup, sugar, glucose, acetic acid, malic acid, fruit juice, Eucommia ulmoides Oliver extract, jujube extract, licorice extract and the like.

The present invention also provides a method of preventing or treating sleep disorder, including a step of administering a composition including a Maydis stigma extract as an active ingredient to a subject.

The present invention also provides a use of a composition for preventing or treating sleep disorder including a Maydis stigma extract.

Hereinafter, the present invention will be described in detail through examples. The following examples are only intended to specifically illustrate the present invention, and it will be apparent to those skilled in the art that the scope of the present invention is not limited by these examples.

### EXAMPLES

Preparation of Maydis Stigma Extract

The Maydis stigma extract used in the present invention was supplied from the Korea Plant Extract Bank of the Korea Research Institute of Bioscience and Biotechnology and was used in experiments, and the Maydis stigma extract of the present invention is a herbal medicine sample that is extracted by a boiling pot at 100° C. for 2 hours and 30 minutes after mixing corn silk and 1 L of water. The extracted material was filtered using non-fluorescent cotton as filter paper and was dried using a vacuum freeze dryer (Biotron, Clean-vac 12) at −70° C. for 24 hours. The dried extract was stored in a −4° C. cold chamber as an original sample.

### Experimental Example 1

Confirmation of Effect of Increase in Melatonin Receptor Expression by Maydis Stigma Extract (FIG. 1a and FIG. 1b)

After treating 2 μg/mL of the Maydis stigma extract to neurons that were isolated from the brain of a 17.5-day-old pregnant mouse fetus and cultured for 7 days, RNA extraction was performed using TRIzol after 24 hours. The isolated RNA was quantified, and RT-PCR was performed to synthesize 0.5 μg cDNA. Changes in melatonin receptor expression were determined using PCR.

As a result, it was shown that for the melatonin receptor 1 (MT1), melatonin receptor expression was increased by 10 times compared to a control group, and it was confirmed that the expression of melatonin receptor 2 (MT2) was increased by 3 times (FIG. 1).

### Experimental Example 2

Confirmation of Sleep-Inducing and Sleep-Promoting Effects by Maydis Stigma Extract in Experimental Animals (FIG. 2)

Experimental animals were 3-week-old ICR mice, and 8 mice were prepared for each group as a negative control group (only drinking water), an experimental group (administered with Maydis stigma) and a positive control group (administered with Valeriana fauriel Briquet). 3-week-old mice were administered with 10 mg/kg of the Maydis stigma extract or 10 mg/kg of the Valeriana fauriel Briquet for one week by oral administration method. After one week, mice in the experimental groups and the control groups were sleep-induced by intraperitoneally injecting 100 mg/kg of ketamine. Sleep onset time and sleep duration time were determined by considering that white mice treated with ketamine were asleep if they showed the belly and were flipped, and white mice were completely awake from sleep if the white mice that showed the belly and were flipped changed the posture showing the back again.

As a result, sleep onset time was 30 seconds shorter on average in the Maydis stigma-treating group compared to the negative control group, and sleep time was about 400 seconds longer in the Maydis stigma-treating group compared to the negative control group, and thus, the sleep-inducing effect and sleep duration time increasing effect of the Maydis stigma extract were confirmed (FIG. 2).

### Experimental Example 3

Confirmation of Toxicity of Maydis Stigma Extract in Experimental Animals (FIGS. 3A and 3B)

By administering water, the Maydis stigma extract and the Valeriana fauriel Briquet to 3-week-old ICR mice for 7 days by oral administration method, weight changes during the treatment period were determined. After 7 days following the sleep-inducing experiment, brain, lung, heart, thymus, liver, kidney, spleen and seminal glands were extracted, and changes in organ weights were measured to determine toxicity of the Maydis stigma extract.

As a result, it was confirmed that there was no weight change between three groups of the negative control group, the Maydis stigma extract administration group and the Valeriana fauriel Briquet administration group (FIG. 3A). After 7 days, brain, lung, heart, thymus, liver, kidney, spleen and seminal glands were extracted from 4-week-old mice and changes in organ weights were measured, and as a result, it was confirmed that there was no change in organ weights between the groups, and thus it was confirmed that there was no toxicity due to the Maydis stigma extract (FIG. 3B).

### Experimental Example 4

Confirmation of Neuron Protective Effect after Pretreating Maydis Stigma Extract (FIG. 4)

After treating 0.2 μg/mL, 2 μg/mL and 20 μg/mL of the Maydis stigma extract for 24 hours to neurons (nerve cell) that were isolated from the brain of a 17.5-day-old pregnant mouse fetus and cultured for 7 days, 50 μM of H2O2 was treated and culture was performed for 24 hours to determine the effect of the Maydis stigma extract on cell death by MTT analysis.

As a result, neurons were killed by H2O2 in the control group which was not treated with the Maydis stigma extract, but in the experimental group that was pretreated with the Maydis stigma extract and treated with H2O2, cell death was suppressed as the concentration of the Maydis stigma extract was increased (FIG. 4).

### Experimental Example 5

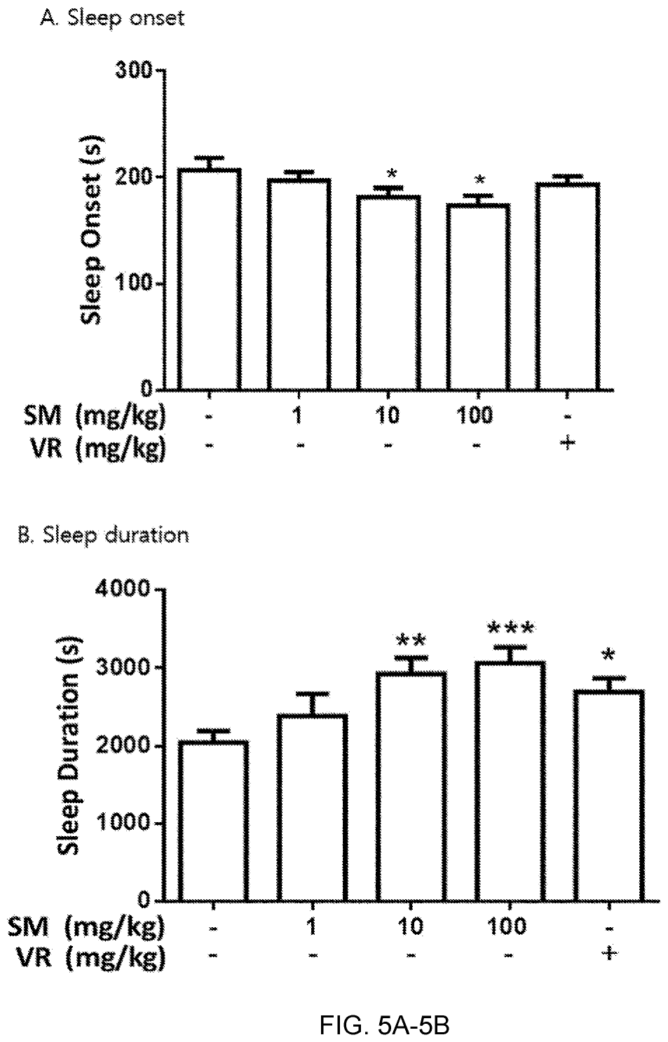

Confirmation of Sleep-Inducing and Sleep-Promoting Effects Depending on Concentrations of Maydis Stigma Extract in Experimental Animals (FIG. 5)

Experimental animals were 3-week-old ICR mice, and 10 mice were prepared for each group as a negative control group (only drinking water), an experimental group (administered with Maydis stigma) and a positive control group (administered with Valeriana fauriel Briquet). 3-week-old mice were administered with 1 mg/kg, 10 mg/kg and 100 mg/kg of the Maydis stigma extract or 10 mg/kg of the Valeriana fauriel Briquet for one week by oral administration method. After one week, mice in the experimental groups and the control groups were sleep-induced by intraperitoneally injecting 42 mg/kg of pentobarbital. Sleep onset time and sleep duration were determined by considering that mice treated with pentobarbital were asleep if they showed the belly and were flipped, and mice were completely awake from sleep if the mice that showed the belly and were flipped changed the posture showing the back again.

As a result, sleep onset time was 20 seconds shorter at 10 mg/kg and 40 seconds shorter at 100 mg/kg in the Maydis stigma treating groups compared to the negative control group, and sleep time was about 900 seconds longer at 10 mg/kg and 1,100 seconds longer at 100 mg/kg in the Maydis stigma treating groups compared to the negative control group, and thus, the sleep-inducing effect and sleep duration time increasing effect of the Maydis stigma extract were confirmed.

### Experimental Example 6

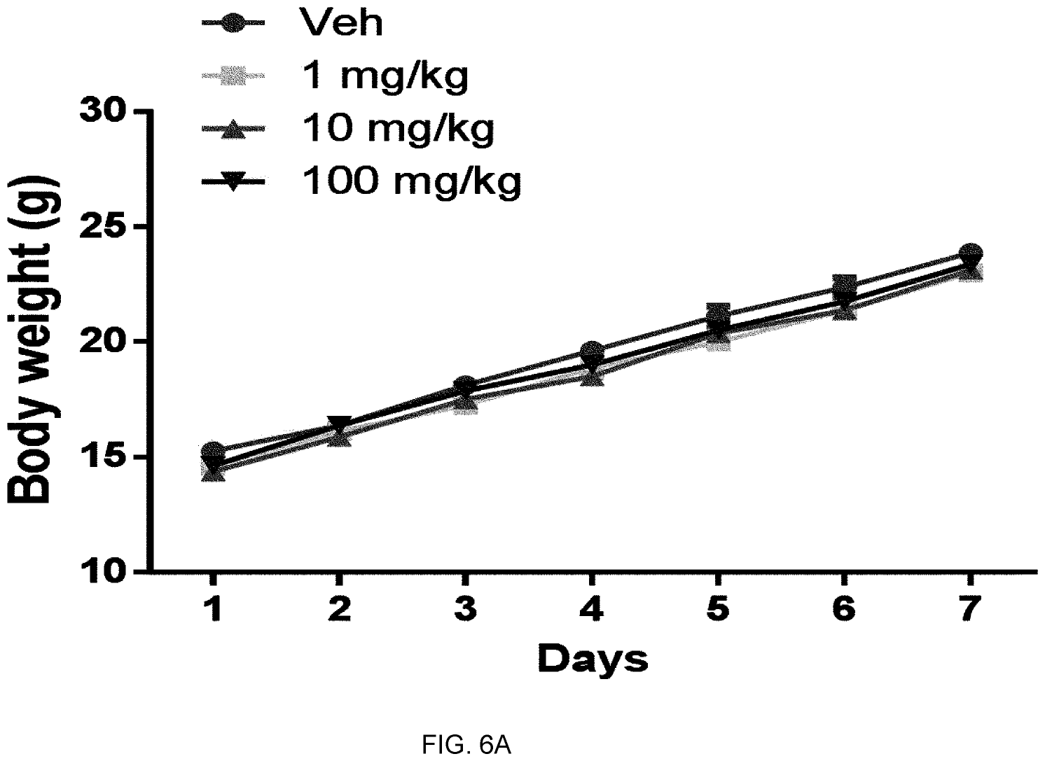

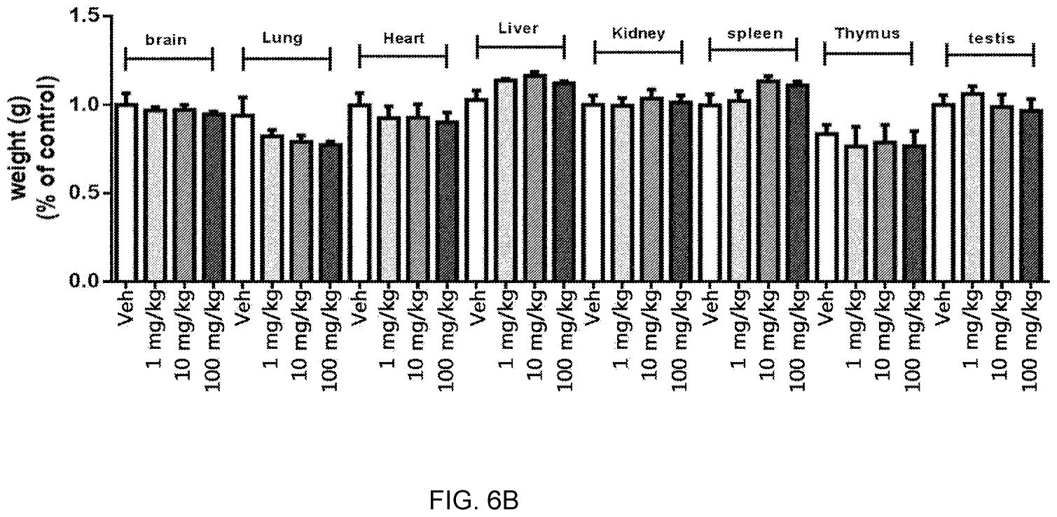

Confirmation of Toxicity Depending on Concentrations of Maydis Stigma Extract in Experimental Animals (FIGS. 6A and 6B)

By administering water, 1 mg/kg, 10 mg/kg and 100 mg/kg of the Maydis stigma extract or 10 mg/kg of the Valeriana fauriel Briquet to 3-week-old mice for 7 days by oral administration method, weight changes during the ingestion period were determined. After 7 days following the sleep-inducing experiment, brain, lung, heart, thymus, liver, kidney, spleen and seminal glands were extracted, and changes in organ weights were measured to determine toxicity of the Maydis stigma extract.

As a result, it was confirmed that there was no weight change between three groups of the negative control group, the Maydis stigma extract administration group and the Valeriana fauriel Briquet administration group (FIG. 6A). After 7 days, brain, lung, heart, thymus, liver, kidney, spleen and seminal glands were extracted from 4-week-old white mice and changes in organ weights were measured, and as a result, it was confirmed that there was no change in organ weights between the groups, and thus it was confirmed that there was no toxicity due to the Maydis stigma extract (FIG. 6B).

### Experimental Example 7

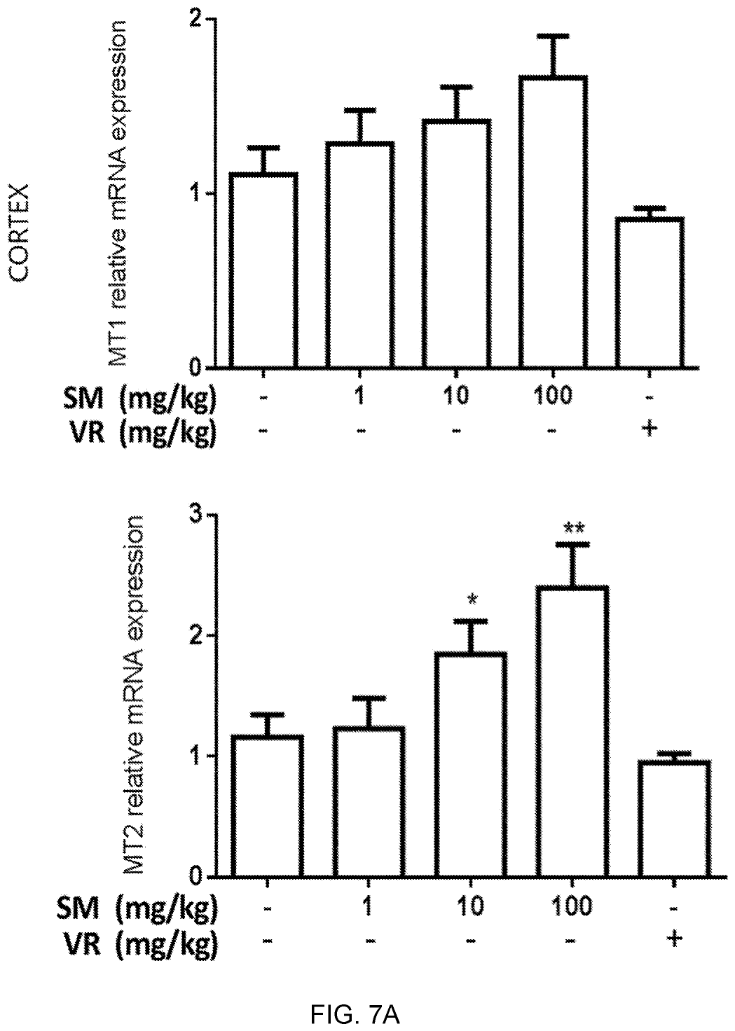

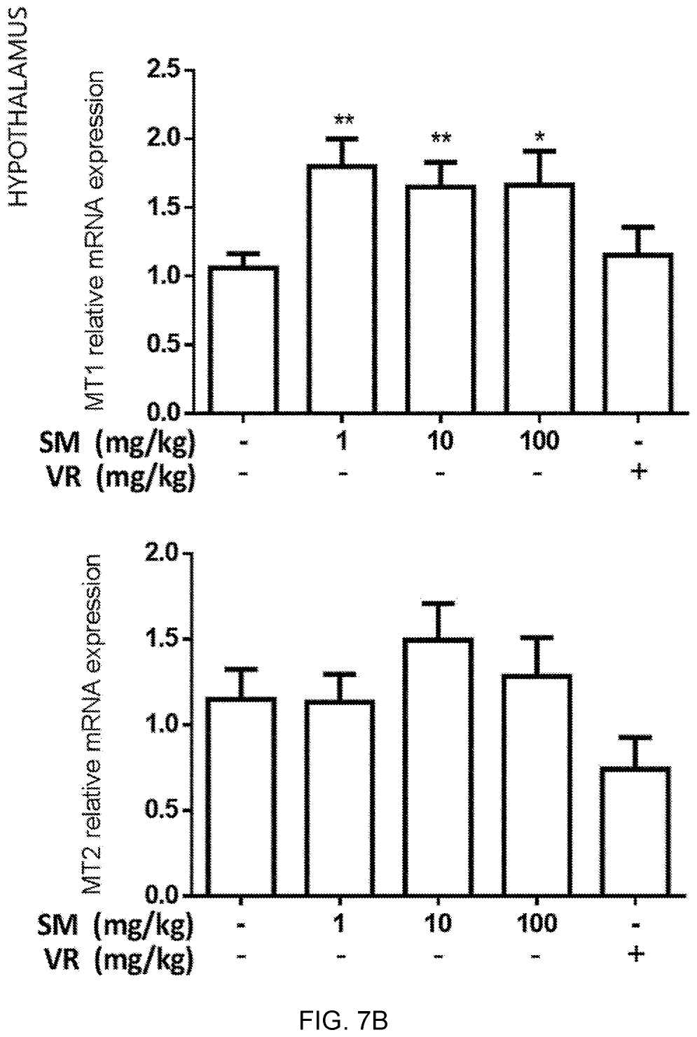

Confirmation of Changes in Melatonin Receptor Expression Depending on Concentrations of Maydis Stigma Extract in Experimental Animals (FIGS. 7A and 7B)

4-week-old male ICR mice were used and were orally administered for one week with 1 mg/kg, 10 mg/kg and 100 mg/kg of the Maydis stigma extract, 10 mg/kg of Valeriana fauriel Briquet to a positive control group and the same volume of distilled water to a negative control group. After anesthetizing by ether, mouse brain was extracted, cerebral cortex and hypothalamus were isolated, and RNA extraction was performed using TRIzol. The isolated RNA was quantified, and RT-PCR was performed to synthesize cDNA. PCR was performed using melatonin receptor 1/2 primer, and changes in melatonin receptor 1/2 expression were determined.

As a result, it was confirmed that the expression of melatonin receptor 1/2 (MT1/2) was increased depending on concentrations of the Maydis stigma extract in cerebral cortex compared to the control groups, and in particular, in case of MT2, it was further increased by about 2.5 times at 100 mg/kg of the Maydis stigma extract compared to the control groups (FIG. 7A). In case of hypothalamus, it was confirmed that the expression of melatonin receptor 1/2 (MT1/2) was increased depending on concentrations of the Maydis stigma extract, and in case of MT1 receptor, it was increased by about 2 times (FIG. 7B).

### Experimental Example 8

Confirmation of Sleep-Inducing and Sleep-Promoting Effects by Maydis Stigma Extract in Sleep Deprivation Model

Experimental animals were 3-week-old ICR mice, and 10 mice were prepared for each group as a negative control group (only drinking water), an experimental group (administered with Maydis stigma) and a positive control group (administered with Valeriana fauriel Briquet). 3-week-old mice were ingested with 1 mg/kg, 10 mg/kg and 100 mg/kg of the Maydis stigma extract or 10 mg/kg of Valeriana fauriel Briquet for one week by oral administration method. After one week, the mice in the experimental groups and the control groups were intraperitoneally injected with 10 mg/kg of caffeine for sleep deprivation and were administered with 42 mg/kg of pentobarbital after 30 minutes to induce sleep. Sleep onset time and sleep duration were determined by considering that white mice treated with pentobarbital were asleep if they showed the belly and were flipped, and mice were completely awake from sleep if the mice that showed the belly and were flipped changed the posture showing the back again.

As a result, it was found that sleep onset time was increased by about 50 seconds in the caffeine-treated group compared to the negative control group, and sleep duration was decreased by 1,000 seconds compared to the negative control group. Compared to the caffeine-treated group, the Maydis stigma extract ingesting group fell asleep about 20 seconds faster at 1 mg/kg, about 30 seconds faster at 10 mg/kg and 40 seconds faster at 100 mg/kg in a concentration-dependent manner, and sleep time was increased in a concentration-dependent manner in the Maydis stigma extract ingesting group compared to the sleep-deprived group, and they were shown to sleep about 450 seconds longer at 100 mg/kg, and thus it was confirmed that there were also a sleep-inducing effect and a sleep time increasing effect by the Maydis stigma extract in the sleep deprivation model by caffeine (FIG. 8).

Although embodiments of the present disclosure are described above, the spirit of the present disclosure is not limited to the embodiments presented in the present specification, and although those skilled in the art may provide other embodiments through the addition, change, or removal of components within the scope of the same spirit of the present disclosure, such embodiments are also included in the scope of the spirit of the present disclosure.

What is claimed is:

1. A pharmaceutical composition for preventing or treating sleep disorder, comprising a Maydis stigma extract as an active ingredient.

2. The pharmaceutical composition of claim 1, wherein the Maydis stigma extract increases the expression of a melatonin receptor.

3. The pharmaceutical composition of claim 1, wherein the Maydis stigma extract decreases sleep onset time or increases sleep duration time.

4. The pharmaceutical composition of claim 1, wherein the Maydis stigma extract is an extract of water, an organic solvent, or a mixture thereof of Maydis stigma.

5. A health functional food composition for preventing or alleviating sleep disorder, comprising a Maydis stigma extract as an active ingredient.

6. The health functional food composition of claim 5, wherein the Maydis stigma extract increases the expression of a melatonin receptor.

7. The health functional food composition of claim 5, wherein the Maydis stigma extract decreases sleep onset time or increases sleep duration time.

8. The health functional food composition of claim 5, wherein the Maydis stigma extract is an extract of water, an organic solvent, or a mixture thereof of Maydis stigma.

9. A method of preventing or treating sleep disorder, comprising a step of administering a composition comprising a Maydis stigma extract as an active ingredient to a subject.

10. The method of claim 9, wherein the Maydis stigma extract increases the expression of a melatonin receptor.

11. The method of claim 9, wherein the Maydis stigma extract decreases sleep onset time or increases sleep duration time.

12. The method of claim 9, wherein the Maydis stigma extract is an extract of water, an organic solvent, or a mixture thereof of Maydis stigma.

13. A use of a composition for preventing and treating sleep disorder comprising a Maydis stigma extract as an active ingredient.

14. The use of claim 13, wherein the Maydis stigma extract increases the expression of a melatonin receptor.

15. The use of claim 13, wherein the Maydis stigma extract decreases sleep onset time or increases sleep duration time.

16. The use of claim 13, wherein the Maydis stigma extract is an extract of water, an organic solvent, or a mixture thereof of Maydis stigma.
